(19) United States
(12) Patent Application Publication
(10) Pub. No.: US 2002/0032420 A1
(43) Pub. Date: Mar. 14, 2002

(54) Perspiration guard

(76) Inventor: Brian Anthony Taylor, Clementon, NJ (US)

Correspondence Address: Matthew J. Peirce, Esq., 1550 Starlight Canyon Avenue, Las Vegas, NV, 89123, US

(21) Appl. No.: 09/941,989

(22) Filed: Aug. 30, 2001

Related U.S. Application Data
(60) Provisional application No. 60/231,201, filed on Sep. 8, 2000.

(51) Int. Cl. A61F 13/15; A61F 13/20; A41D 27/12

(52) U.S. Cl. 604/359; 604/385.03; 604/385.05; 604/387; 2/53; 2/56

(57) ABSTRACT

A new and improved apparatus is disclosed that will absorb sweat from a user's underarms, thereby preventing staining of a user's clothing and removing most, if not all, of the odor present that is associated with underarm sweat. The present invention would comprise an oval-shaped pad that would include a straight sticky pad that would traverse right down the middle of the oval pad. Preferably, the straight sticky pad would travel along the long dimension of the oval-shaped pad. The pad would preferably be approximately ⅛ of an inch thick. The pads would preferably include an impregnated scent which would be heat-activated. In addition, the present invention would include a clear plastic sticky backing to ensure that moisture from a person who would sweat heavily would not soak through and stain or wet a user's clothes.

### BACKGROUND OF THE INVENTION

The present invention concerns that of a new and improved apparatus that will absorb sweat from a user's underarms, thereby preventing staining of a user's clothing and removing most, if not all, of the odor present that is associated with underarm sweat.

### DESCRIPTION OF THE PRIOR ART

U.S. Pat. No. 4,856,111, issued to Sholes, discloses a disposable perspiration shield generally for use with an upper torso garment utilizing a first layer of adsorbent tissue which lies next to a second layer of adsorbent material.

U.S. Pat. No. 4,663,780, issued to Rawlings et al., discloses an invention which relates to the absorbtion of body odor and is concerned with a provision of a pad which can be incorporated into a garment at the armhole soye to absorb odor.

### SUMMARY OF THE INVENTION

The present invention concerns that of a new and improved apparatus that will absorb sweat from a user's underarms, thereby preventing staining of a user's clothing and removing most, if not all, of the odor present that is associated with underarm sweat. The present invention would comprise an oval-shaped pad that would include a straight sticky pad that would traverse right down the middle of the oval pad. Preferably, the straight sticky pad would travel along the long dimension of the oval-shaped pad. The pad would preferably be approximately ⅛ of an inch thick. The pads would preferably include an impregnated scent which would be heat-activated. In addition, the present invention would include a clear plastic sticky backing to ensure that moisture from a person who would sweat heavily would not soak through and stain or wet a user's clothes.

There has thus been outlined, rather broadly, the more important features of a new and improved apparatus for absorbing sweat and preventing staining of clothes in order that the detailed description thereof that follows may be better understood and in order that the present contribution to the art may be better appreciated. There are, of course, additional features of the new and improved apparatus for absorbing sweat and preventing staining of clothes that will be described hereinafter and which will form the subject matter of the claims appended hereto.

In this respect, before explaining at least one embodiment of the new and improved apparatus for absorbing sweat and preventing staining of clothes in detail, it is to be understood that the new and improved apparatus for absorbing sweat and preventing staining of clothes is not limited in its application to the details of construction and to the arrangements of the components set forth in the following description or illustrated in the drawings. The new and improved apparatus for absorbing sweat and preventing staining of clothes is capable of other embodiments and being practiced and carried out in various ways. Also, it is to be understood that the phraseology and terminology employed herein are for the purpose of descriptions and should not be regarded as limiting.

As such, those skilled in the art will appreciate that the conception, upon which this disclosure is based, may readily be utilized as a basis for the designing of other structures, methods and systems for carrying out the several purposes of the present new and improved apparatus for absorbing sweat and preventing staining of clothes. It is important, therefore, that the claims be regard as including such equivalent constructions insofar as they do not depart from the spirit and scope of the present invention.

It is therefore an object of the present invention to provide a new and improved apparatus for absorbing sweat and preventing staining of clothes which has all of the advantages of the prior art and none of the disadvantages.

It is another object of the present invention to provide a new and improved apparatus for absorbing sweat and preventing staining of clothes which may be easily and efficiently manufactured and marketed.

It is another object of the present invention to provide a new and improved apparatus for absorbing sweat and preventing staining of clothes which is of durable and reliable construction.

It is yet another object of the present invention to provide a new and improved apparatus for absorbing sweat and preventing staining of clothes which is economically affordable and available to the buying public.

It is yet another object of the present invention to provide a new and improved apparatus for absorbing sweat and preventing staining of clothes which provides additional benefits not present in the prior art.

Other objects, features and advantages of the present invention will become more readily apparent from the following detailed description of the preferred embodiment when considered with the attached drawings and appended claims.

### BRIEF DESCRIPTION OF THE DRAWINGS

FIG. 1 shows a top view of the present invention.

FIG. 2 shows a perspective view of the present invention.

### DESCRIPTION OF THE PREFERRED EMBODIMENT

A priority date of Sep. 8, 2000, is hereby claimed due to the filing of provisional application No. 60/231,201 on Sep. 8, 2000.

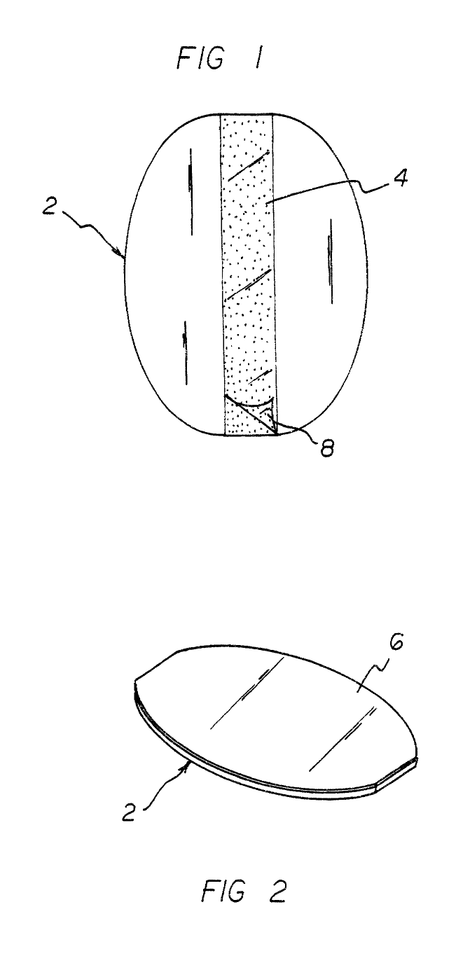

FIG. 1 shows a top view of the present invention. The present invention concerns that of a new and improved apparatus that will absorb sweat from a user's underarms, thereby preventing staining of a user's clothing and removing most, if not all, of the odor present that is associated with underarm sweat. The present invention would comprise an oval-shaped pad 2 that would include two sides, a first side and a second side. Each pad 2 would include a straight sticky attachment 4 that would traverse right down the middle of each pad 2 on the front side of each pad 2. The pad 2 would have a length and a width, with the length being the longer of the two dimensions.

Preferably, the straight sticky attachment 4 would travel along the length, or the longer dimension, of the oval-shaped pad on each front side of each pad 2. The pad would preferably be approximately ⅛ of an inch thick. The pads would preferably include an impregnated scent which would be heat-activated. The impregnated scent would only be released once a user would place on his or her clothing and then proceed to wear this clothing, at which time, the user's body heat would activate and release the impregnated scent. Straight sticky attachment 4 would be covered by covering 8, which would protect straight sticky attachment 4 until a user would want to use a particular pad 2.

FIG. 2 shows a perspective view of the present invention. In this figure, the present invention can be seen to include a clear plastic sticky backing 6 to ensure that moisture from a person who would sweat heavily would not soak through and stain or wet a user's clothes. Backing 6 would be attached to the portion of the first side of each pad 2 that would not be covered by straight sticky attachment 4. Backing 6 would ensure that the sweat from a user who would sweat heavily would not seep through the entire pad 2 and soak into a user's clothes, causing stains and smells.

To use the present invention, a user would apply pad 2 with the straight sticky attachment 4 down in the armpit area of a shirt or other top garment. Each pad 2 would have to be applied on the inner side of the piece of clothing. Once in place, each pad 2 should absorb substantially all or all of a user's odors and stains that would result from underarm sweat.

What I claim as my invention is:

1. An apparatus for absorbing sweat and preventing staining of clothes comprising:
(a) an oval pad having a length and a width, the oval pad also having two ends, a first end and a second end, the oval pad also having two sides, a front side and a rear side, the oval pad being about one-eighth of an inch in thickness, the oval pad being fabricated from a water absorbing material,
(b) a sticky attachment strip attached to the front side of the oval pad, the strip having two ends, a first end and a second end, the first end of the sticky attachment strip located at the first end of the oval pad, the second end of the sticky attachment strip located at the second end of the oval pad,
(c) a covering located on top of the sticky attachment strip,
(d) a backing located on the front side of the oval pad on the areas of the front side of the oval pad where the sticky attachment strip is not located,
(e) whereby a user would remove the covering and place the sticky attachment strip against an inside portion of a user's clothing in the general location of an armpit, whereby the user would then put on the piece of clothing.

2. An apparatus for absorbing sweat and preventing staining of clothes according to claim 1 wherein the oval pad further includes a chemical that releases a heat-activated impregnated scent upon coming into contact with a heat source.

3. An apparatus for absorbing sweat and preventing staining of clothes according to claim 2 wherein the heat source would be a human body.
